(19) United States
(12) Patent Application Publication
(10) Pub. No.: US 2004/0236347 A1
(43) Pub. Date: Nov. 25, 2004

(54) Trocar sheath tube

(75) Inventor: Hitoshi Karasawa, Hachioji-shi (JP)

Correspondence Address: FRISHAUF, HOLTZ, GOODMAN & CHICK, P.C., 25th Floor, 767 Third Avenue, New York, NY, 10017-2023, US

(73) Assignee: Olympus Optical Co., Ltd., Tokyo (JP)

(21) Appl. No.: 10/869,228

(22) Filed: Jun. 15, 2004

Related U.S. Application Data
(63) Continuation of application No. 09/521,367, filed on Mar. 8, 2000.

(30) Foreign Application Priority Data
Mar. 12, 1999 (JP) 11-066368
Feb. 22, 2000 (JP) 2000-044548

(51) Int. Cl. A61M 25/00

(52) U.S. Cl. 606/108

(57) ABSTRACT

A trocar sheath tube according to the present invention comprises an insert portion introduced into the inside of a body, the insert portion having a tube passage, the tube passage being capable of passing a medical instrument internally, the insert portion guiding the medical instrument into the inside of a boy through this tube passage, a holding portion provided at the proximal end side of the insert portion in order to hold the proximal end side of the medical instrument inserted through the insert portion, the holding portion having an opening communicating with the tube passage of the insert portion, a sealing member removably mounted to the opening of the holding portion, the sealing member having a first sealing portion coming into intimate contact with the medical instrument, the sealing member sealing a space between the medical instrument and the holding portion by this first sealing portion, and a fixing member adopted to fix the sealing member to the holding portion.

### CROSS-REFERENCE TO RELATED APPLICATIONS

This application is based upon and claims the benefit of priority from the prior Japanese Patent Applications No. 11-066368, filed Mar. 12, 1999; and No. 2000-044548, filed Feb. 22, 2000, the entire contents of which are incorporated herein by reference.

### BACKGROUND OF THE INVENTION

The present invention relates to a trocar sheath tube that is percutaneously inserted into the inside of a body and functions as a guide tube for guiding an optical viewing tube (or an endoscope) performing observation of a lesion and a treatment instrument for performing treatment of the lesion into the inside of a body.

A conventional trocar sheath tube, as disclosed in Japanese Utility Model Registration No. 2533624, for example, consists of an elongated insert portion having a tube passage capable of passing an optical viewing tube or a treatment instrument into the inside of a body and a holding portion provided at the proximal end side of this insert portion. The insert portion is percutaneously inserted into the inside of the body with a trocar internal needle. The holding portion has a main body section connected to the proximal end side of the insert portion; a cover air-tightly connected to the main body section; and a rubber cap air-tightly fitted to the cover. A first hole into which the optical viewing tube or treatment instrument can be inserted is provided at the inside of the cover. A flap valve biased in a direction in which the hole is closed by a spring and a packing for air-tightly holding an interval between the cover and the flap valve are provided inside of the cover. The rubber cap consists of an elastic material, and is removably fitted with the periphery of an inlet of the first hole of the cover in a contact state. A second hole into which the optical viewing tube or treatment instrument can be inserted is provided at the rubber cap. This second hole and the first hole are substantially coaxially positioned with these holes being spaced with each other along the axial direction of the insert portion. With such construction, when the optical viewing tube or treatment instrument is introduced into the inside of the cover through the first and second holes, and is inserted into the insert portion so as to push away the flap valve, the second hole of the rubber cap comes into intimate contact with the optical viewing tube or treatment instrument, and air tightness between one of these tube and instrument and the trocar sheath tube is ensured.

In addition, in the trocar sheath tube disclosed in Jpn. Pat Appln. KOKAI Publication No. 5-293112 and Jpn. UM Appln. KOKOKU Publication No. 4-34802, two air-tight valves are coupled with each other via a band-shaped coupling member capable of being elastically deformed. In this case, the hole diameter of one air-tight valve differs from the hole diameter of the other air-tight valve.

In the meantime, in the trocar sheath tube disclosed in Japanese Utility Model Registration No. 2533624, the packing comes into firmly contact with the flap valve, and is positioned deeply at the recess inside of the cover opposed to the flap valve in order to hold an interval between the cover and the flap valve air-tightly. Therefore, it is difficult to wash the packing and the periphery of the packing. In addition, in the case where the packing is damaged by wash brush during surgery or during preoperative or postoperative handling or in the case where the packing is hardened or deformed by repetitive washing and sterilization of the sheath tube, it is required to repair and replace the packing because air tightness between the cover and the flap valve cannot be maintained. However, in this case also, the packing is positioned deeply at the recess inside of the cover, thus making it difficult to repair or replace the packing. In contrast, the rubber cap merely fitted with the cover can be easily repaired and replaced, and however, the cap may be removed by being caught by a thick forceps or stepped forceps while these forceps are inserted and removed during surgery.

On the other hand, the trocar sheath tube disclosed in Jpn. Pat Appln. KOKAI Publication No. 5-293112 and Jpn. UN Appln. KOKOKU Publication No. 4-34802 has a problem that the air-tight valve swings in no-load state. That is, a coupling member for coupling the two air-tight valves with each other is exploded and flatly expanded by its elasticity in no-load state, and these two air-tight valves are greatly spaced each other. In this state, since one air-tight valve is coupled with the fixed other air-tight valve by elasticity of the coupling member, the one air-tight valve swings, and is not defined at its position, making it difficult to perform intra-operative mounting operation of the air-tight valve.

### BRIEF SUMMARY OF THE INVENTION

A first object of the present invention is to provide a trocar sheath tube in which a sealing member can be easily washed and replaced, and maintenance can be easily performed at a low cost. In addition, a second object of the present invention is to provide a trocar sheath tube in which a sealing member is hardly removed, operability of mounting the sealing member is proper, and the number of parts is reduced.

These objects of the present invention are achieved by the following trocar sheath tube. That is, the trocar sheath tube according to the present invention comprises: an insert portion introduced into the inside of a body, the insert portion having a tube passage, the tube passage being capable of passing a medical instrument internally, the insert portion guiding the medical instrument into the inside of a body through this tube passage; a holding portion provided at the proximal end side of the insert portion in order to hold the proximal end side of the medical instrument inserted into the insert portion, the holding portion having an opening communicating with a tube passage of the insert portion; and a sealing member removably mounted to the opening of the holding portion, the sealing member having a first sealing portion coming into intimate contact with the medical instrument, the sealing member for sealing a space between the medical instrument and the holding portion by means of this first sealing portion; and a fixing member being adopted to fix the sealing member to the holding portion.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

FIG. 1 is a side view showing a trocar sheath tube according to a first embodiment of the present invention;

FIG. 2 is a sectional view showing the trocar sheath tube shown in FIG. 1;

FIG. 3 is an enlarged sectional view showing a holding portion of the trocar sheath tube shown in FIG. 1;

FIG. 4 is an enlarged sectional view showing the holding portion in a sealing mode different from that shown in FIG. 3;

FIG. 5 is a sectional view showing a push button mounting portion of the trocar sheath tube shown in FIG. 1;

FIG. 6 is a side view showing a state in which the holding portion of the trocar sheath tube shown in FIG. 1 is dissembled into the main body section and the cover section;

FIG. 7 is a front view showing a cover section viewed in a direction indicated by arrow 7 shown in FIG. 6;

FIG. 8A is a sectional view taken along the line 8A-8A shown in FIG. 6;

FIG. 8B is a sectional view showing a state in which the cover section is rotated from the state shown in FIG. BA;

FIG. 9 is a sectional view showing a state in which a seal fixing frame is removed from the holding portion of the trocar sheath tube shown in FIG. 1;

FIG. 10 is a side view showing a cover section of the holding portion of the trocar sheath tube shown in FIG. 1;

FIG. 11 is a front view showing a seal fixing frame viewed in a direction indicated by arrow 11 shown in FIG. 9;

FIG. 12 is a front view showing a seal receiving portion of the trocar sheath tube shown in FIG. 1 viewed from the proximal end side;

FIG. 13 is a sectional view showing a sealing member of the trocar sheath tube shown in FIG. 1;

FIG. 14 is a perspective view showing a first modified example of the sealing member;

FIG. 15A is a sectional view showing a second modified example of the sealing member;

FIG. 15B is a perspective view showing a second modified example of the sealing member;

FIG. 16 is a view showing distortion force acting with an arm section of the sealing member shown in FIGS. 15A and 15B;

FIG. 17A is a sectional view showing a third modified example of the sealing member;

FIG. 17B is a plan view showing a sealing member viewed in a direction indicated by arrow 17B of FIG. 17A;

FIG. 18A is a sectional view showing a fourth modified example of the sealing member;

FIG. 18B is a sectional view taken along the line 18B-18B of FIG. 18A;

FIG. 19 is a sectional view showing a fifth modified example of the sealing member;

FIG. 20 is a sectional view showing a sixth modified example of the sealing member;

FIG. 21 is a side view showing a trocar sheath tube according to a second embodiment of the present invention;

FIG. 22 is a sectional view taken along the line 22-22 of FIG. 23;

FIG. 23 is a plan view showing a trocar sheath tube shown in FIG. 21;

FIG. 24 is a sectional view taken along the line 24-24 shown in FIG. 21;

FIG. 25 is a sectional view showing a state in which a sealing member is separated from the holding portion of the trocar sheath tube shown in FIG. 21;

FIG. 26 is a side view showing a holding section in a state in which the sealing member is removed;

FIG. 27A is a sectional view taken along the line 27A-27A shown in FIG. 26;

FIG. 27B is a sectional view corresponding to that shown in FIG. 27A, which shows a state in which a button section of a modified ring is pressed;

FIG. 28 is a side view showing a state in which the holding portion of the trocar sheath tube shown in FIG. 21 is dissembled into the main body section and the cover section;

FIG. 29 is a sectional view showing the holding portion of the trocar sheath tube shown in FIG. 21;

FIG. 30 is a front view showing the main body section of the holding portion of the trocar sheath tube shown in FIG. 21, viewed from the proximal end side;

FIG. 31 is a front view showing the cover section of the holding portion of the trocar sheath tube shown in FIG. 21, viewed from the tip end side;

FIG. 32A is a schematic sectional view showing a mounting portion in a state in which the cover is mounted to the main body section of the holding portion of the trocar sheath tube shown in FIG. 21;

FIG. 32B is a schematic sectional view showing a state in which the cover section is rotated against the main body section from the state shown in FIG. 32A;

FIG. 33 is a sectional view showing the trocar sheath tube shown in FIG. 21; and

FIG. 34 is a sectional view showing the trocar sheath tube in a sealing mode different from that shown in FIG. 33.

### DETAILED DESCRIPTION OF THE INVENTION

Hereinafter, embodiments of the present invention will be described with reference to the accompanying drawings.

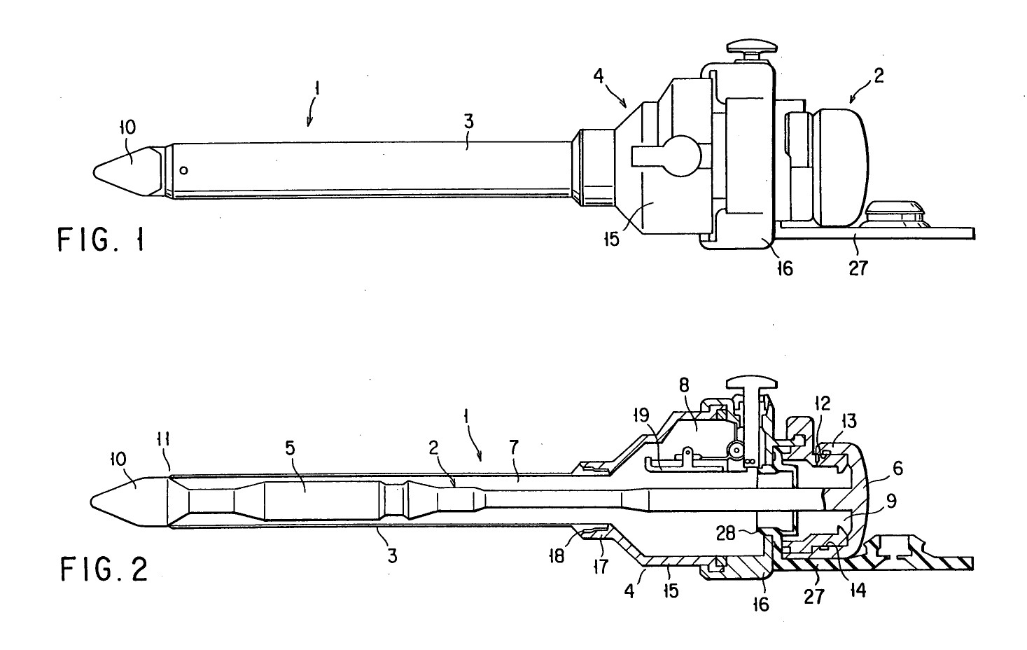

FIGS. 1 to 13 show a first embodiment of the present invention. FIGS. 1 and 2 show a state in which a trocar internal needle 2 is assembled with a trocar sheath tube 1 according to the present embodiment. As shown in FIGS. 1 and 2, the sheath tube 1 has an elongated insert portion 3 at a tip end side, and has a holding portion 4 at a proximal end side. The internal needle 2 has an elongated needle portion 5 at a tip end side, and has an internal needle main body section 6 at a proximal end side. The insert portion 3 of the sheath tube 1 has a tube passage 7 opened at both ends, and the proximal end side of the tube passage 7 communicates with an internal space 8 of the holding portion 4. The tube passage 7 can pass a medical instrument such as endoscope, treatment instrument or internal needle 2 internally. The insert portion 3 is introduced into the inside of a body, and can guide the medical instrument into the inside of a body through the tube passage 7. The holding portion 4 is provided at the proximal end side of the insert portion 3 in order to hold the proximal end side of the medical instrument inserted into the insert portion 3.

A hole 9 communicating with an internal space 8 of the holding portion 4 is provided at the proximal end side of the holding portion 4. A sharp blade portion 10 is formed at the tip end of the internal needle 2. When the internal needle 2 is inserted into the insert portion 3 through the hole 9, and the internal needle main body section 6 is fixed to the proximal end side of the holding portion 4, the needle portion 5 of the internal needle 2 reaches the inside of the tube passage 7 through the internal space 8, and the blade portion 10 is exposed from an opening 11 of the insert portion 3 to the outside.

An elastic ring 13 is incorporated into the inside of a tip end side opening 12 of the internal needle main body section 6. When this ring 13 is fitted into a peripheral groove 14 provided in the vicinity of the proximal end side of the holding portion 4, axial movement of the internal needle main body section 6 is restricted against the holding portion 4, and the internal needle main body section 6 is fixed to the holding portion 4. The holding portion 4 consists of a main body section 15 and a cover section 16. The main body section 15 is removably fixed to the proximal end of the insert portion 3 by means of a screw 17 fixed to the tip end. An O-ring 18 consisting of an elastic material provided adjacent to the screw 17 ensures air tightness between the insert portion 3 and the holding portion 4.

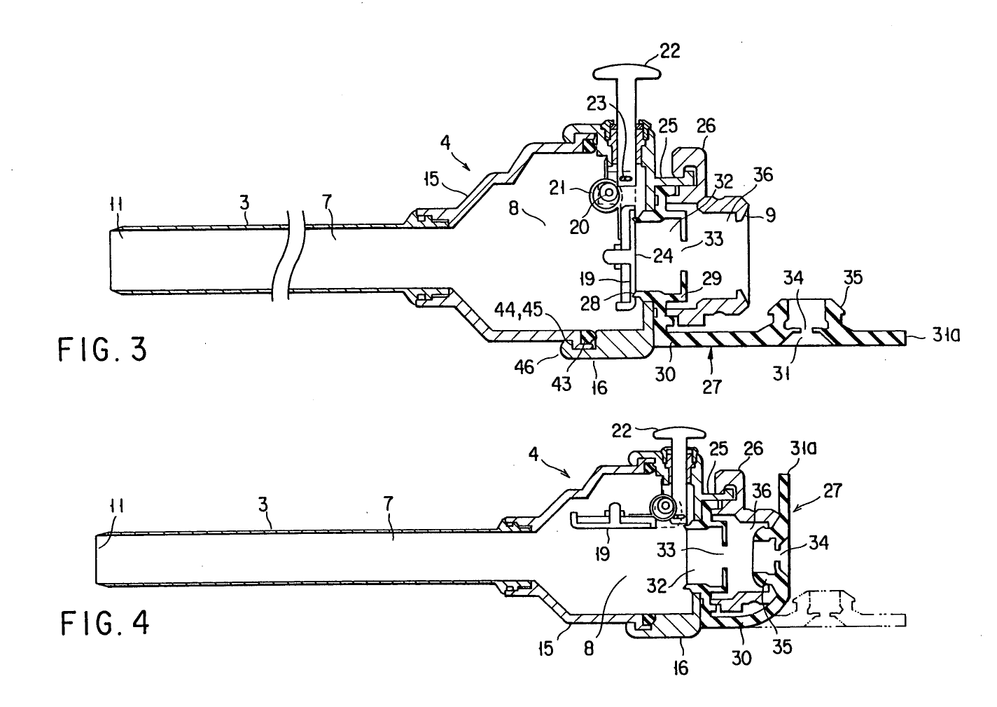

As shown in FIG. 3, a flap valve 19 is provided as an opening/closing valve inside of a cover section 16. This flap valve 19 can be rotated between a first position (refer to FIG. 4) where the flat valve 19 is parallel to the longitudinal axial direction of the sheath tube 1 and a second position (refer to FIG. 3) where the flat valve 19 is orthogonal to the longitudinal axial direction of the sheath tube 1 about a rotary shaft 20 extending in a direction orthogonal to the longitudinal axial direction of the sheath tube 1. In addition, the flap valve 19 is always biased to the second position by means of a spiral spring 21 spirally held around the rotary shaft 20.

A push button 22 capable of sliding in a direction orthogonal to the longitudinal axial direction of the sheath tube 1 is provided on a side wall of the cover section 16. In addition, a pin 23 engaged with the push button 22 is provided at the flap valve 19. With this construction, when the push button 22 is operated to be pushed, rotational force acts with the flap valve 19 via the pin 23. Then, the flap valve 19 is rotated from the second position (refer to FIG. 3) to the first position (refer to FIG. 4) about the rotary shaft 21 against the biasing force of the spring 21. In addition, the flap valve 19 has a back face 24 exposed to the outside facing to the hole 9. When the back face 24 is pressed by the tip end of the internal needle 2 to be introduced into the holding portion 4 or the tip end of the forceps or the like (not shown), the flap valve 19 is rotated from the second position (refer to FIG. 3) to the first position (refer to FIG. 4) about the rotary shaft 21 against the biasing force of the spring 21.

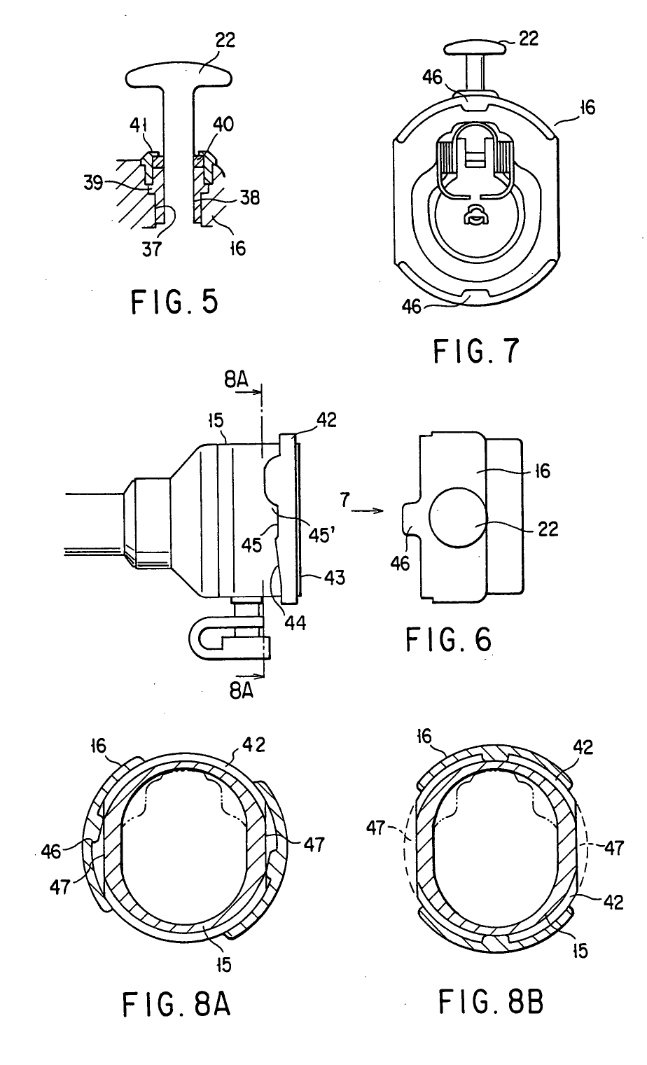

A structure of the mounting portion of the cover section 16 to which the push button 22 is to be mounted is shown in FIG. 5. As illustrated, a through hole 37 extending in a direction orthogonal to the longitudinal axial direction of the sheath tube 1 is formed on the side wall of the cover section 16. A cylindrical sleeve 38 is fitted into this through hole 37. The sleeve 38 is positioned against the cover section 16 by means of a jaw portion 39. A ring-shaped packing 40 consisting of an elastic material is engaged into the through hole 37 on the sleeve 38. The packing 40 is fixed by means of a stop nut 41 screwed against the cover section 16. The sleeve 38 is formed by a resin material such as polysulfone, polyether ether sulfone or polyphenyl sulfone with proper sliding properties and abrasion proof properties.

A seal receiving portion 25 is formed at the proximal end side of the cover section 16. A sealing member 27 consisting of an elastic material is sandwiched between this seal receiving portion 25 and a seal fixing frame 26 as a fixing member to be fitted therewith, and is fixed. The sealing member 27 has a sealing cap portion 29 as a first sealing portion at its proximal end side, and a cylindrical sealing ring portion 28 as a second sealing portion at its tip end side. In addition, a small-diameter sealing portion 31 as a third sealing portion is coupled with the sealing cap portion 29 via an arm portion 30 extending by forming a right angle therewith (extending along the longitudinal axial direction of the insert portion 3 in a state (shown in FIG. 3) in which the sealing member 27 is mounted to the seal receiving portion 25 of the holding portion 4).

When the sealing member 27 is completely mounted to the seal receiving portion 25, the tip end of the sealing ring portion 28 protrudes in an internal space 8 of the holding portion 4. At this time, the tip end of the sealing ring portion 28 push the flap valve 19 into the internal space 8 against the biasing force of the spring 21, and comes into pressure contact with the flap valve 19 by means of the biasing force of the spring 21. In this manner, air tightness in the internal space 8 is ensured. From this state, when the flap valve 19 is rotated against the biasing force of the spring 21, an internal space 32 of the sealing ring portion 28 communicates with the internal space 8 of the holding portion 4.

A large-diameter hole 33 communicating with the internal space 32 of the sealing ring portion 28 is provided at the center of the sealing cap portion 29. When a medical instrument such as internal needle 2 or forceps (not shown) is inserted through this large-diameter hole 33, an internal face of the sealing cap portion 29 forming the large-diameter hole 33 comes into intimate contact with the peripheral face of the medical instrument, and a space between the medical instrument and the holding portion 4 is sealed. That is, the internal space 32 of the sealing ring portion 28 is sealed to the outside.

A short diameter hole 34 whose internal diameter is smaller than that of the large-diameter hole 33 and a ring-shaped mounting jaw portion 35 are provided at a small-diameter sealing portion 31. When an arm portion 30 is elastically bent, and a mounting jaw portion 35 is pushed into a mounting hole 36 at the proximal end of the seal fixing frame 26, as shown in FIG. 4, the large-diameter hole 33 and the small-diameter hole 34 are arranged in line along the longitudinal axial direction of the insert portion 3. When a medical instrument (such as forceps) whose diameter is smaller than the internal diameter of the large-diameter hole 33 is inserted into the small-diameter hole 34, the small-diameter sealing portion 31 comes into intimate contact with the peripheral face of this medical instrument, and ensures air tightness. The arm portion 30 of the sealing member 27 extends to the proximal end side along a direction orthogonal to the extending direction of the proximal end face of the cover section 16. In a state in which the sealing member 27 is mounted to the seal receiving portion 25, the tip end of the arm portion 30 is brought into intimate contact with the proximal end face of the cover section 16. A lip portion 31a is formed at the end of the arm portion 30 extending from the small-diameter sealing portion 31 to the proximal end side. When this lip portion 31a is gripped by hand, and the sealing member 27 is pulled, the mounting jaw portion 35 can be removed from the mounting hole 36.

A state in which the holding portion 4 of the sheath tube 1 is divided into the main body section 15 and the cover section 16 is shown in FIG. 6. A flange portion 42 and a packing ring 43 are provided at the proximal end of the main body section 15. At an end face of the tip end side of the flange 42, a cam face 44 and a recess portion 45 are provided at the upper and lower sections symmetrical to the center axis of the main body section 15. On the other hand, as shown in FIG. 7, at the tip end of the cover section 16, claw portions 46 are provided at the upper and lower sections symmetrical to the center axis of the cover section 16. In a state in which the main body section 15 and the cover section 16 are assembled with each other, as shown in FIG. 3, they are biased by means of the packing ring 43 intervened between both parties in a direction spaced from each other. By this biasing force, the claw portion 46 is pressed against the cam face 44 or the recess portion 45, and is meshed.

As shown in FIGS. 8A and 8B, a vertically-cut blank portion 47 is formed at the right and left of the flange portion 42. As shown in FIG. 8A, when the cover section 16 is rotated against the main body section 15, and the claw portion 46 is positioned at the blank portion 47, the main body section 15 and the cover section 16 are able to be approached each other, and the claw portion 46 can be advanced over the flange 42. When the cover section 16 is rotated against the main body section 15 from the state shown in FIG. 8A, the claw portion 46 slides on the cam face 44, and drops in the recess portion 45. Rotation of the cover section 16 against the main body section 15 is restricted by means of a stopper face 45′ formed adjacent to the recess portion 45. That is, the cover section 16 cannot be rotated to the rotated position where the claw portion 46 has been over the recess portion 45.

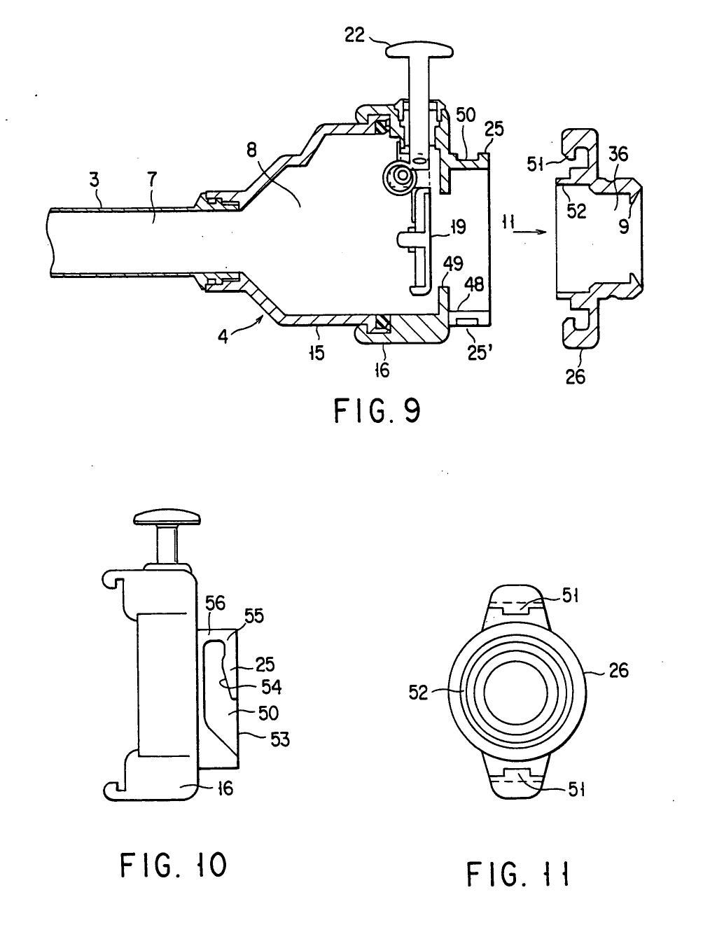

FIG. 9 shows a state in which the sealing member 27 is removed by removing the seal fixing frame 26 from the cover section 16. As is evident from this figure, a cylindrical space 48 for receiving the sealing member 27 and a window portion 49 for partitioning the cylindrical space 48 from the internal space 8 of the holding portion 4 are provided at the seal receiving portion 25 of the cover section 16.

As shown in FIGS. 10 and 11, on the peripheral face of the seal receiving portion 25 of the cover section 16, cam grooves 50 are provided at the upper and lower sections symmetrical to the center axis of the cover section 16. On the other hand, on the seal fixing frame 26, claw portions 51 are provided at the upper and lower sections symmetrical to the center axis thereof. In addition, a cylindrical rim 52 to be brought into pressure contact with the sealing member 27 is provided at the tip end of the seal fixing frame 26. In a state in which the claw portions 51 are positioned at an opening end 53 of the cam groove 50, while the cover section 16 and the seal fixing frame 26 are approached each other, when the seal fixing frame 26 is rotated against the cover section 16, the claw sections 51 slide on the cam face 54 present at the proximal end side of the cam groove 50, reaches the recess portion 55, and stops in abutment with a stopper face 56 vertically provided at its tip.

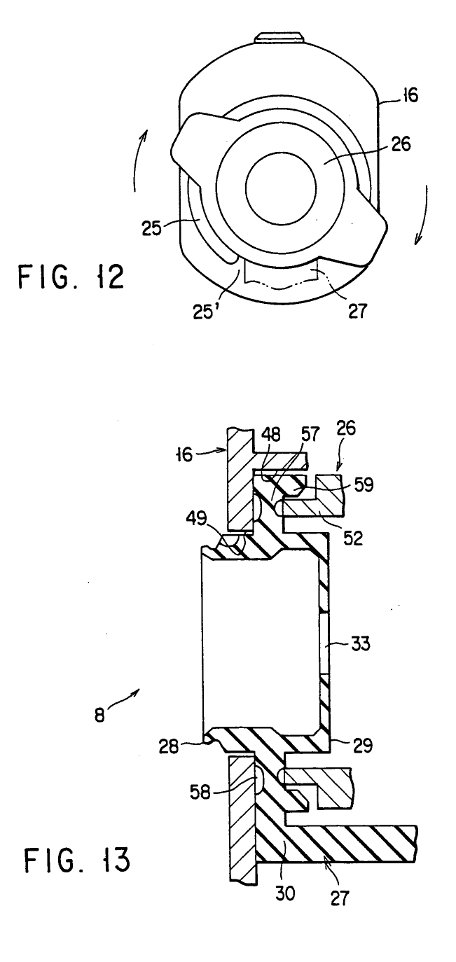

As shown in FIG. 13, when a disk portion 57 of the sealing member 27 is dropped into the cylindrical space 48 of the cover section 16, the sealing ring portion 28 protrudes into the internal space 8 of the holding portion 4 through the window portion 49 of the cover section 16. At this time, as shown in FIG. 12, the arm portion 30 of the sealing member 27 is led out to the outside of the holding portion 4 through a cutout portion 25′ formed at the seal receiving portion 25. In this state, as described previously, when the claw portions 51 of the seal fixing frame 26 is positioned at the cam groove 50 of the cover section 16, and the seal fixing frame 26 is rotated against the cover section 16, the cylindrical rim 52 of the seal fixing frame 26 comes into pressure contact with the disk portion 57 of the sealing member 27, and the sealing member 27 is fixed to the holding portion 4. At this time, repulsion force from the sealing member 27 caused by the seal fixing frame 26 pressing the sealing member 27 works in a direction in which the cover section 16 and the seal fixing frame 26 are spaced from each other, and the claw portions 51 are pressed against the cam face 54 or the recess portion 55, and is rigidly meshed with each other. At the disk portion 57 of the sealing member 27, a groove 58 having its arch-shaped section is provided on a face opposite to a face with which the cylindrical rim 52 comes into contact. This groove 58 is provided in annular shape over the full periphery of the sealing member 27, and elasticity of the disk portion 57 is enhanced. In addition, a cylindrical wall portion 59 is provided at the periphery of the disk portion 57. This wall portion 59 is caught by the cylindrical rim 52 when external force is applied to the sealing member 27, and the disk portion 57 is deformed, and the disk portion 57 is prevented from slipping off from the seal receiving portion 25.

According to the sheath tube 1 of the present embodiment that has been described above, the following advantageous effects can be achieved.

(1) The sealing member 27 is removably mounted to the holding portion 4, and is rigidly fixed by the seal fixing frame 26. Therefore, the sealing member 27 or the periphery of the mounting portion of the cover section 16 on which this sealing member is mounted is easily washed. In addition, in the case where the sealing member 27 is damaged and degraded, the sealing member 27 is easily replaced.

(2) The sealing ring portion 28 coming into intimate contact with the flap valve 19 to ensure air tightness and the sealing cap portion 29 coming into intimate contact with a medical instrument such as optical viewing tube or forceps to ensure air tightness are integrally formed. Therefore, the number of parts is reduced, and maintenance is facilitated. In addition, replacement cost can be reduced.

(3) The mounting hole 36 provided at the seal fixing frame 26 is arranged coaxially with the large-diameter hole 33 of the sealing cap portion 29 of the sealing member 27, and the inclination of the medical instrument such as optical viewing tube or treatment instrument to be inserted through the large-diameter hole 33 is restricted. Therefore, a gap is not provided between the large-diameter hole 33 of the sealing cap portion 29 and the medical instrument, and air tightness can be well ensured during surgery.

(4) The arch-shaped groove 58 is provided at the disk portion (mounting portion) 57 of the sealing member 27. Thus, the elasticity of this portion is increased, and the sealing member 27 can be fixed with small force by means of the seal fixing frame 26. In addition, the sealing member 27 is hardly removed after it is mounted.

(5) The arm portion 30 of the sealing member 27 extends in a direction orthogonal to the proximal end face of the cover section 16 when the sealing member is mounted. That is, the small-diameter sealing portion 31 as a third sealing portion is coupled with the sealing cap 29 via an arm portion 30 extending by forming a right angle therewith (extending along the longitudinal axial direction of the insert portion 3 in a state (shown in FIG. 3) in which the sealing member 27 is mounted to the seal receiving portion 25 of the holding portion 4). Therefore, even if axial force is applied to the sealing member 27 during surgery, the sealing member 27 is not removed from the cylindrical rim 52 of the seal fixing frame 26.

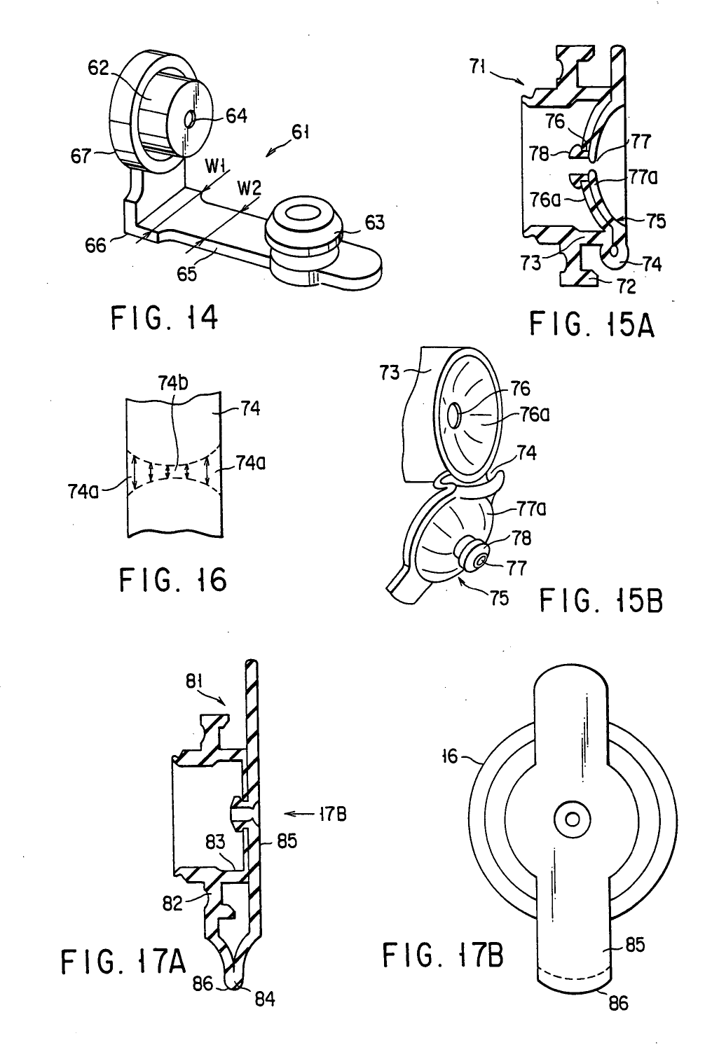

FIG. 14 shows a first modified example of a sealing member. A sealing member 61 according to this modified example consists of an elastic material, and comprises a sealing cap portion 62 (a first sealing portion) mounted in the holding portion 4 of the sheath tube 1; and a small-diameter sealing portion (a third sealing portion) 63 coupled with the sealing cap portion 62 by means of the arm portion 65. A large-diameter hole 64 with its relatively large diameter is provided at the sealing cap portion 62, and a small-diameter hole (not shown) with its internal diameter smaller than the large-diameter hole 64 is provided at the small-diameter sealing portion 63.

A connection portion 66 between the sealing cap portion 62 and the arm portion 65 is bent at a right angle. That is, the arm portion 65 extends in a direction orthogonal to a mounting face 67 of the cap portion 62 coming into intimate contact with the sealing sheath tube 1. When the arm portion 65 is elastically bent, the small-diameter sealing portion 63 can be easily mounted on the mounting hole 36 of the seal fixing frame 26 at the proximal end of the sheath tube 1. A width W1 of the connection portion 66 is set to be greater than a width W2 of the arm portion 65.

With such construction, the small-diameter sealing portion 63 is provided at the arm portion 65 extending in a direction orthogonal thereto from the sealing cap portion 62, and the axial center of the sealing cap portion 62 and the axial center of the small-diameter sealing portion 63 are substantially orthogonal to each other. Thus, the arm portion 65 is merely bent by about 90 degrees, whereby the small-diameter sealing portion 63 can be easily mounted to the mounting hole 36 of the seal fixing frame 26. In addition, the width W1 of the connection portion 66 is set to be greater than the width W2 of the arm portion 65. Thus, the rigidity of the connection portion 66 is increased, and the arm portion 65 is free from being inclined or swung against the mounting face 67. Therefore, the small-diameter sealing portion 63 is maintained to be stable at the same position, and position check of the small-diameter sealing portion 63 can be performed speedily during in use. As a result, mounting operation of the small-diameter sealing portion 63 is facilitated. There is achieved the same effect when the rubber hardness of the connection portion 66 is increased and molded, and a reinforce member is mounted to the connection portion 66.

FIGS. 15A, 15B and 16 show a second modified example of a sealing member. As illustrated, a sealing member 71 according to this modified example consists of a mounting portion 72 mounted in the holding portion 4 of the sheath tube 1; a sealing cap portion 73; an arm portion 74; and a small-diameter sealing portion 75 provided at the tip end of the arm portion 74. A cap-shaped (substantially hemisphere shaped) support face 76a is provided at the proximal end of the sealing cap portion 73, and a large-diameter hole 76 with its relatively large internal diameter is provided at the center of the support face 76a. A cap-shaped support face 77a having the same curvature as the support face 76a is provided at a small-diameter sealing portion 75. This support face 77a is formed to be slightly smaller than the support face 76a, and has the small-diameter hole 77 with its internal diameter smaller than the hole 76; and a mounting flange 78 at its center.

As shown in FIG. 15B, the arm portion 74 is provided at the outer rim of the proximal end of the sealing cap portion 73, and has the same curvature as the peripheral shape of the sealing cap portion 73. Thus, when the arm portion 74 has this curvature, the arm portion 74 is stabilized in a state in which the arm portion is folded as shown in FIG. 15A or in a state in which the arm portion is folded back at an angle between 180 degrees and 360 degrees on an opposite side as shown in FIG. 15B. On the other hand, in a state in which the arm portion 74 is opened flatly by 180 degrees, that is, in a state in which the arm portion is in intermediate state between the state shown in FIG. 15A and the state shown in FIG. 15B, the expansion quantity of a rim portion 74a and the expansion quantity of a center portion 74b is significantly different from each other as shown in FIG. 16, and distortion force is generated, making it impossible to maintain its original mode. That is, in no-load state, the sealing member 71 enters either of modes, i.e., a state in which the arm portion 74 is folded, and a mounting flange 78 is overlapped with the large-diameter hole 76 as shown in FIG. 15A or a state in which the arm portion 74 is folded back at an angle between 180 degrees and 360 degrees, the small-diameter sealing portion 75 is completely distant from the sealing cap portion 73, and the hole 76 is completely exposed. As shown in FIG. 15A, when the arm portion 74 is folded, the mounting flange 78 can be easily fitted into the large-diameter hole 76 by the elasticity of the arm portion 74 with slight pushing force from a position opposite to the large-diameter hole 76.

According to such construction, the small-diameter sealing portion 75 is free from being swung at its unstable position, and there can be quickly switched a state in which the forceps can be inserted into the large-diameter hole 76 of the sealing cap portion 73 by sliding the small-diameter portion 75 to its side in order to use thick forceps; and a state in which the forceps can be inserted into the small-diameter hole 77 by mounting the small-diameter sealing portion 75 to the sealing cap portion 73 in order to use a forceps which is thinner than the internal diameter of the large-diameter hole 76. In addition, because of its simple structure, washing and sterilization can be performed easily and reliably, is stable, and can be manufactured at a inexpensive cost. Further, two support faces 76a and 77a are formed at the similar curvature, alignment of these faces is facilitated, and a mount state can be checked reliably and easily with the touch of operating fingers.

FIGS. 17A and 17B show a third modified example of a sealing member. As illustrated, a sealing member 81 according to this modified example has a mounting portion 82 mounted at the holding portion 4 of the sheath tube 1; a sealing cap portion 83; and an arm portion 84. A small-diameter sealing portion 85 is provided at the tip end of the arm portion 84. The arm portion 84 extends from the mounting portion 82 to its radial outside, is folded back at a folding portion 86, and is connected to the small-diameter sealing portion 85. The folding portion 86 forms a curve as shown in FIG. 17B. Thus, when the folding portion 86 has this curvature, when the folding portion 86 is exploded flatly, the arm portion 84 has an expansion quantity at the rim and center portions as in the second modified example, and thus, distortion force is produced, making it Impossible to maintain its original mode. That is, the arm portion 84 is stable in a state the arm portion 84 is completely folded as shown in FIG. 17A (an opening angle is 0 degree) or in a state in which the arm portion is warped by around 360 degrees.

Thus, in the sealing member 81 according to a third modified example, unlike a second modified example, the arm portion 84 extends to the radial outside. Thus, when the small-diameter sealing portion 85 is not used, the small-diameter sealing portion 85 moves to a position distant more significantly than the sealing cap portion 83, and does not block insertion and removal of the forceps or operation thereof.

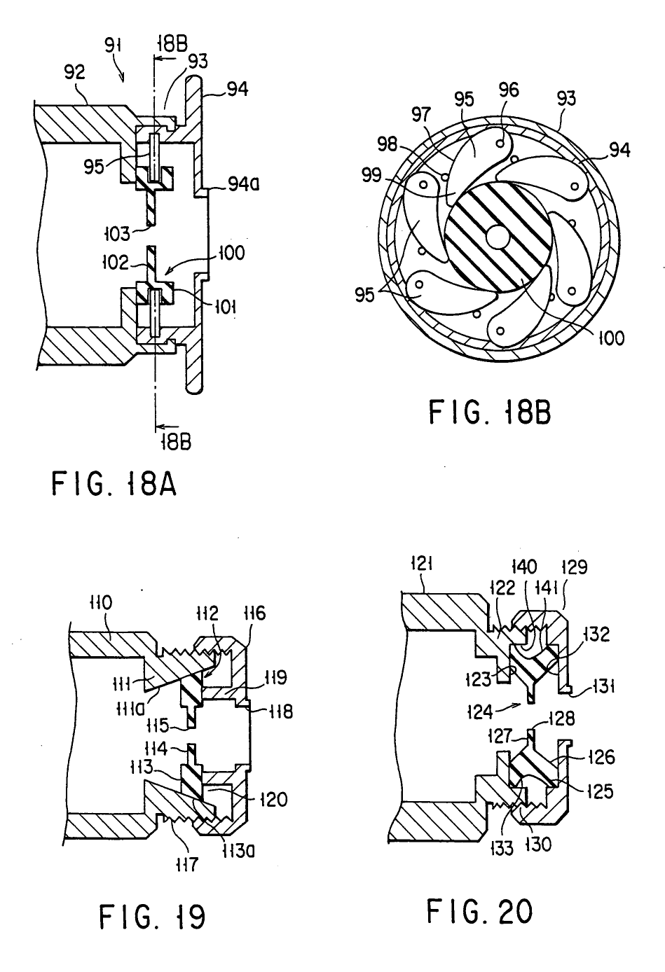

FIGS. 18A and 18B show a fourth modified example of a sealing member. As illustrated, a sealing member according to this modified example is constructed as a valve mechanism mounted to the cover section of the sheath tube. That is, a cylindrical frame 93 is provided at the proximal end of a cover section 92 of a sheath tube 91. A rotary knob 94 and a plurality of blades 95 disposed at equal intervals along the peripheral direction of the rotary knob 94 are provided at the inside of the frame 93. Each blade 95 can rotate about a pin 96 fixed to the rotary knob 94 and serves as a hole diameter changeable member for changing the hole diameter of a sealing valve 100 inside of the blades 95.

In FIG. 18B, when the rotary knob 94 is rotated in counterclockwise direction, a cam face 97 outside of the blade 95 is meshed with a cam pin 98 fixed to the cover section 92 or the frame 93. Thus, the blade 95 is inwardly pushed in the radial direction by means of a cam pin 98, and is swiveled about the pin 96.

In this manner, tip ends 99 of the blade 95 inwardly moving to the radial direction approach each other, comes into pressure contact with the sealing valve 100, compress this valve, and reduces the internal diameter of the sealing valve 100.

In the sealing valve 100, its periphery is formed as a ring-shaped thickness portion 101, and its center is formed as a thinly film-shaped portion 102. A circular hole 103 is provided at the center of a film-shaped portion 102.

With such construction, in a state in which the forceps or internal needle is passed into a hole 103 through an opening 94a provided at the center of the rotary knob 94, when the rotary knob 94 is rotated in counterclockwise direction, its inwardly film-shaped portion 102 is compressed together with compression of the thickness portion 101 due to movement of the blade 95, and the hole 103 is reduced in diameter. In this manner, the film-shaped portion 102 comes into intimate contact with the periphery of the forceps or internal needle, and air tightness is ensured. Conversely, the rotary knob 94 is rotated in clockwise direction from this state, the tip ends 99 of each blade 95 are distant from each other. Contraction force does not act with the thickness portion 101, and the hole 103 is restored in its original size.

The sealing valve with such construction can function as the flap valve 19 and the sealing member 27 according to the first embodiment. In addition, the structure is easier than that of these members 19 and 27, and moreover, is hardly damaged. In addition, the number of parts is reduced, assembling is facilitated, and manufacturing cost is reduced. Further, the diameter of the hole 103 can be arbitrarily reduced (controlled). Thus, there is no need for mounting and removing the small-diameter sealing portion 31 according to the thickness of the forceps unlike in the first embodiment, and intra-operative replacement of the forceps can be performed speedily.

FIG. 19 shows a fifth modified example of a sealing member. As illustrated, a sealing member according to this modified example is constructed as a valve mechanism mounted to the cover section of the sheath tube. That is, a cylindrical frame 111 is provided at the proximal end of a cover section 110 of the sheath tube, and the internal face of this frame 111 is formed as an inwardly convergent tapered face 111a.

A substantially ring-shaped sealing valve 112 is disposed inside of the frame 111. The sealing valve 112 is formed as a ring-shaped thickness portion 113 at its periphery, and is formed as a thin film-like portion 114 at its center. A circular hole 115 is provided at the center of the film-like portion 114. The peripheral face of the thickness portion 113 is formed as a tapered face 113a having the same inclined angle as the tapered face 111a, and comes into contact with the tapered face 111a slidably.

A push-in knob 116 as a hole diameter changeable member is screwed at a screw portion 117 on the proximal end of the frame 111 for connection. The push-in knob 116 has an opening 118 at its center, and has a ring-shaped push-in portion 119 at its tip end. The push-in portion 119 comes into contact with the rear face of the thickness portion 113 of the sealing valve 112.

With such construction, when the push-in knob 116 is screwed against the frame 111, the sealing valve 112 is pressurized by the push-in portion 119. In this manner, the sealing valve 112 is moved to the inside (tip end side) with its convergent diameter along the tapered face 111a. As a result, the thickness portion 113 is compressed inwardly in radial direction by the tapered face 111a, the film-like portion 114 at its inside is also compressed, and the hole 115 is reduced in diameter. At this time, if a forceps or an internal needle is passed into the hole 115 through the opening 118 of the push-in knob 116, the film-like portion 114 comes into intimate contact with the periphery of the forceps or internal needle, and air tightness is ensured. Conversely, when the push-in knob 116 is rotated in reverse direction from this state, and is moved to the proximal end side, the sealing valve 112 is moved to the outside (proximal end side) with its divergent diameter along the tapered face 111a. In this case, a compression force does not act with the thickness portion 113, and the hole 115 is restored to its original size.

A valve mechanism (sealing valve 112) with such construction can function as the flap valve 19 and the sealing member 27 according to the first embodiment, and is simpler in construction than the valve mechanism according to the fourth modified example.

FIG. 20 shows a sixth modified example of a sealing member. As illustrated, the sealing member according to this modified example is constructed as a valve mechanism to be mounted on the cover section of the sheath tube. That is, a cylindrical frame 122 is provided at the proximal end of a cover section 121. At the proximal end side of the frame 122, there are formed a first annular abutment face 123 extending inwardly in radial direction; and a second annular abutment portion 140 extending so as to be orthogonal to the first abutment face 123. A compression knob 129 as a hole diameter changeable member is screwed with a screw portion 130 formed at the peripheral face of the proximal end of the frame 122 for connection. An opening 131 is provided at the center of the compression knob 129. In addition, at the inside of the compression knob 129, there are formed a third annular abutment face 132 extending inwardly in radial direction; and a fourth annular abutment face 141 extending so as to be orthogonal to the third abutment face 132. A sealing valve 124 is arranged between the compression knob 129 and the frame 122. In this case, the sealing valve 124 abuts against the first and third abutment faces 123 and 132 on both side faces thereof, and abuts against the second and fourth abutment faces 140 and 141 on its peripheral face. In addition, the sealing valve 124 consists of a ring-shaped thickness portion 125 positioned at its peripheral portion; a tapered migration portion 126 extending so as to be convergent (thinner) toward the radial inside from the thickness portion 125; and a film-like portion 127 extending from the migration portion 126 toward a circular hole 128.

With such construction, when the compression knob 129 is screwed against the frame 122, the thickness portion 125 of the sealing valve 124 is compressed to be sandwiched between the first abutment face 132 of the compression knob 129 and the third abutment face 123 of the frame 122. At this time, the sealing valve 124 is restricted from outward deformation in radial direction by the second and third abutment faces 140 and 141, and the sealing valve 124 is deformed inwardly in radial direction. In this manner, the central proximity of the thickness portion 125 is pushed to the inside, the film-like portion 127 at its inside is compressed, and the hole 128 is reduced in diameter. At this time, when the forceps or internal needle is passed into the hole 128 through the opening 131 of the compression knob 129, the film-like portion 127 comes into intimate contact with the periphery of the forceps or internal needle, and air tightness is ensured. Conversely, when the compression knob 129 is rotated in reverse direction from this state, and is moved to the proximal end side, a compression force does not act with the thickness portion 125, and the hole 128 is restored to its original size. When a V-shaped groove 133 is provided all over the periphery of the thickness portion 125, the thickness portion 125 can be easily deformed inwardly in radial direction.

A valve mechanism (sealing valve 124) with such construction can function as a flap valve 19 and a sealing valve 27 according to the first embodiment, and is simpler in construction than the valve mechanism according to the fourth modified example.

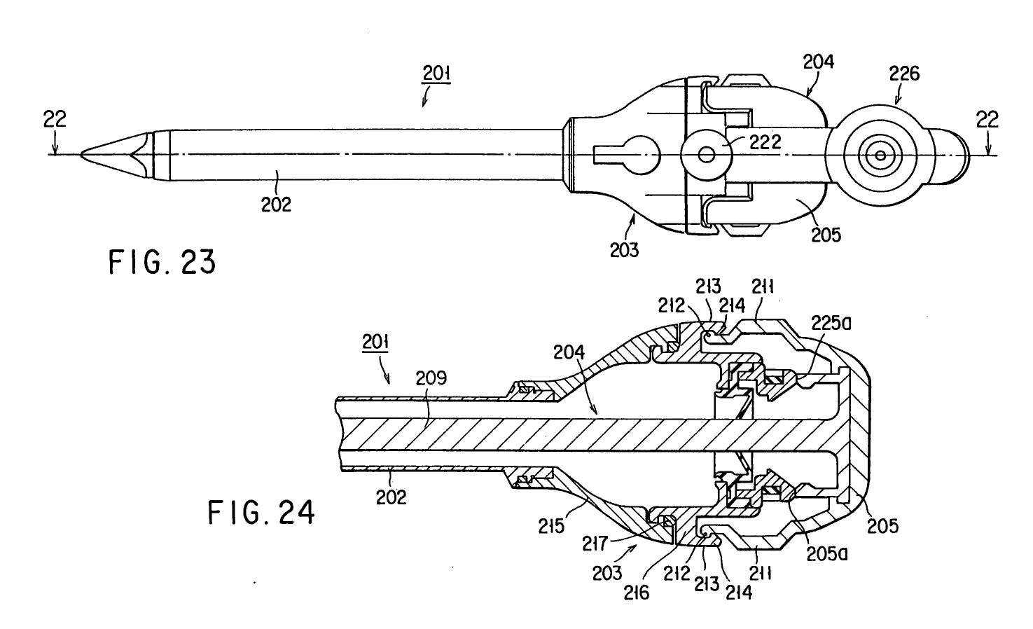

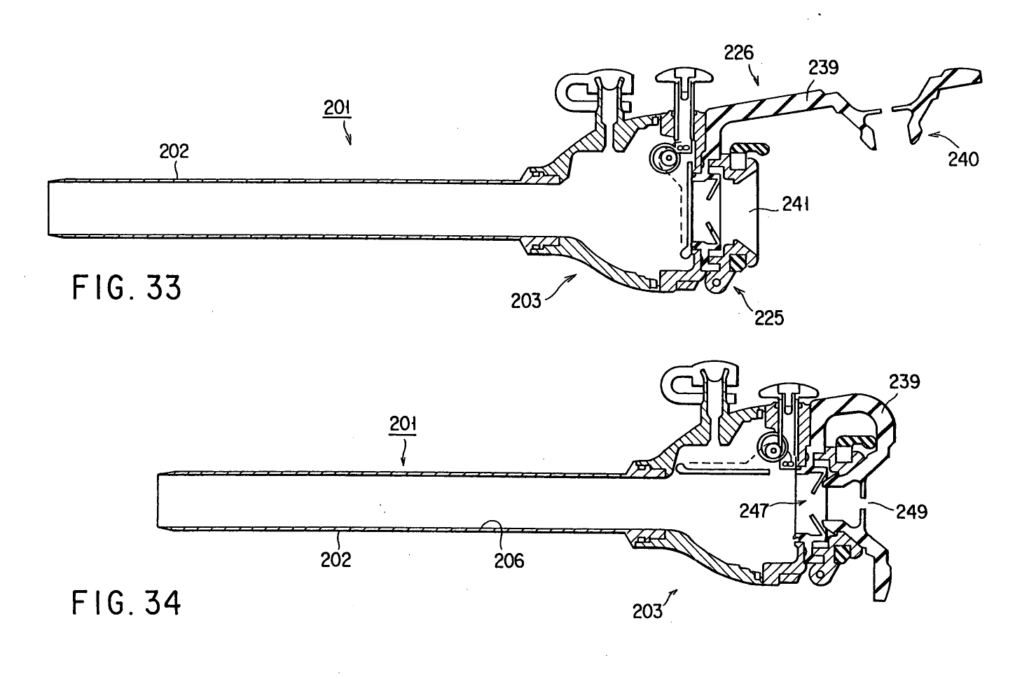

FIGS. 21 to 34 show a second embodiment of the present invention.

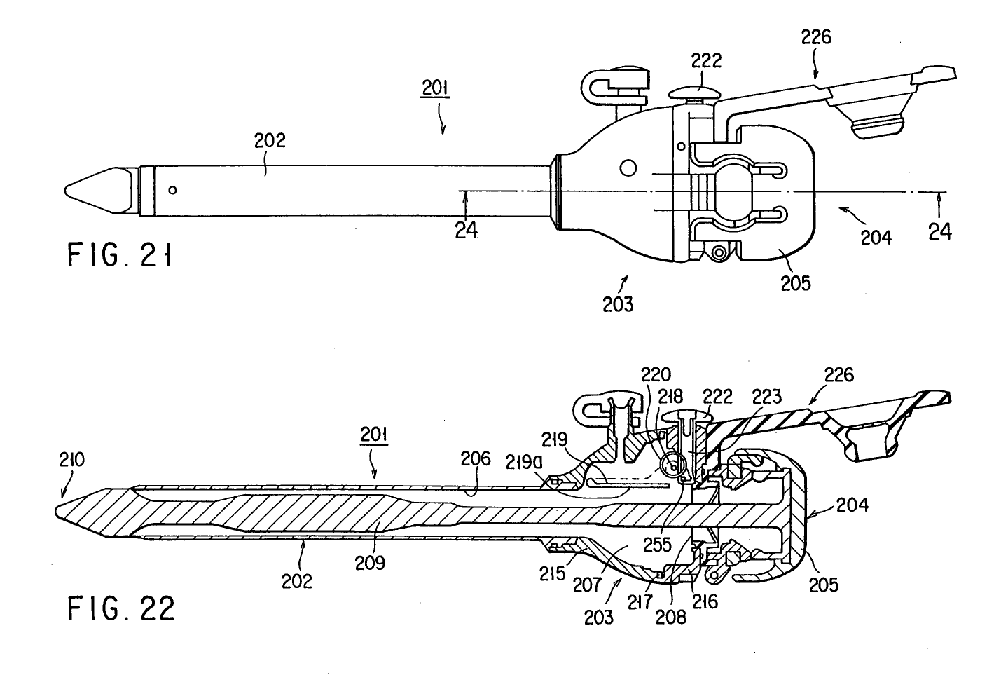

FIGS. 21 and 22 show a state in which a trocar internal needle 204 is assembled with a trocar sheath tube 201 according to the present embodiment. As shown in FIGS. 21 and 22, the sheath tube 201 has an elongated insert portion 202 at its tip end side, and has a holding portion 203 at its proximal end side. The internal needle 204 has an elongated needle portion 209 at a tip end side, and has an internal needle main body 205 at a proximal end side. The insert portion 202 of the sheath tube 201 has a tube passage 206 opened at both ends, and the proximal end side of the tube passage 206 communicates with an internal space 207 of the holding portion 203. Tube passage 206 can pass a medical instrument such as an endoscope, treatment instrument or internal needle 204 internally. The insert portion 202 is introduced into the inside of a body, and can guide the medical instrument into the inside of a body through the tube passage 206. The holding portion 203 is provided at the proximal end side of the insert portion 202 in order to hold the proximal end side of the medical instrument inserted into the insert portion 202. An opening 208 communicating with the internal space 207 is provided at the proximal side of the holding portion 203.

On the other hand, a sharp blade portion 210 is formed at the tip end of the needle portion 209 of the internal needle 204. When the internal needle 204 is inserted into the holding portion 203 through the opening 208, and the internal needle main body section 205 is connected to the holding portion 203, the needle portion 209 of the internal needle 204 reaches the inside of the tube passage 206 through the internal space 207, and the blade portion 210 is protruded (exposed) to the outside from the tip end of the insert portion 202. The internal needle main body section 205 is rotatably connected to the holding portion 203 so as to rotate the needle portion 209 of the internal needle 204 in the sheath tube 201 and arbitrarily change the orientation of the blade 210.

As shown in FIG. 24, the holding portion 203 of the sheath tube 201 consists of a main body section 215 and a cover section 216. Arm portions 211 extending in the tip end direction are provided, respectively, at both sides of the internal needle main body section 205. At tip ends of these arm portions 211, claws 212 protruded to the outside is provided. On the other hand, receiving portions 213 extending in the proximal end direction are provided, respectively, at both sides of the cover section 216 of the holding portion 203. An inwardly projecting claw 214 is provided inwardly at the end of this receiving portion 213. With such construction, when the internal needle main body section 205 is strongly pressed to the cover section 216 of the holding portion 203, the arm portion 211 is inclined inwardly, and the claw 212 is meshed therewith over the claw 214. In order to perform directional positioning, a stopper face 205a facing on the tip end side is formed inwardly of the main body section 205 of the internal needle 204, and a proximal end face 225a abutting against the stopper face 205a is formed at a seal fixing portion 225 (described later) of the holding portion 203.

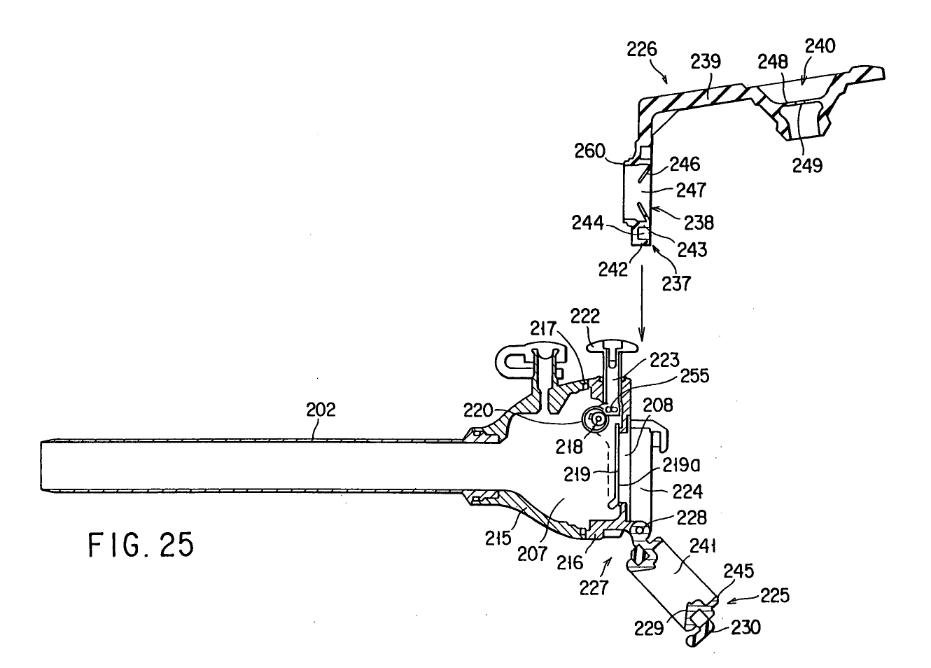

As shown in FIGS. 22 and 25, a flap valve 219 as an opening/closing valve is provided inside of the cover section 216. This flap valve 219 can be rotated at the center of a rotary shaft 218 extending to the direction orthogonal to the longitudinal direction of the sheath tube 201 between a first position (refer to FIG. 22) parallel to the longitudinal axial direction of the sheath tube 201 and a second position (refer to FIG. 25) orthogonal to the longitudinal axial direction of the sheath tube 201. In addition, the flap valve 219 is always biased toward the second position by means of a spiral spring 220 spirally held on the rotary shaft 218. The center of the rotary shaft 218 is eccentrically de-centered from the longitudinal center axis of the sheath tube 201.

A slide shaft 223 capable of sliding in a direction orthogonal to the longitudinal axial direction of the sheath tube 201 is provided on the side wall of the cover section 216. In addition, a push button 222 is provided at the end of the slide shaft 223 protruded from the side wall of the cover section 216. A pin 255 engaged with the slide shaft 223 is provided at the flap valve 219. With this construction, when the slide shaft 223 is operated to be pushed downward via the push button 22, a rotational force acts with the flap valve 219 via the pin 255. Then, the flap valve 219 is rotated about the rotary shaft 218 from the second position (refer to FIG. 25) to the first position (refer to FIG. 22) against the biasing force of the spring 220. In addition, the flap valve 219 has a back face 219a. When the back face 219a is pressed by the tip end of the internal needle 204 introduced into the holding portion 203 or by the tip end of the forceps or the like, the flap valve is rotated about the rotary shaft 218 from the second position (refer to FIG. 25) to the first position (refer to FIG. 22) against the biasing force of the spring 220. The push button 222 is rotatably mounted to the slide shaft 223 so as not to allow a rotational moment to be applied to the flap valve 219 when the push button 222 is rotated.

As shown in FIG. 25, the seal receiving portion 224 and the seal fixing portion 225 as a fixing member are provided at the proximal side of the cover section 216. The sealing member 226 consisting of an elastic member is sandwiched between the seal receiving portion 224 and the seal fixing portion 225. The seal fixing portion 225 is connected to the cover section 216 via a hinge portion 227, and can be rotated between an engaging position where the sealing member 226 is sandwiched between the seal receiving portion 224 and the seal fixing portion 225 and a distant position where the seal fixing portion 225 is spaced from the seal receiving portion 224, about a rotary shaft 228 provided at the cover section 216. Therefore, when the seal fixing portion 225 is rotated in a direction spaced from the seal receiving portion 224, the sealing member 226 can be removed from the seal receiving portion 224.

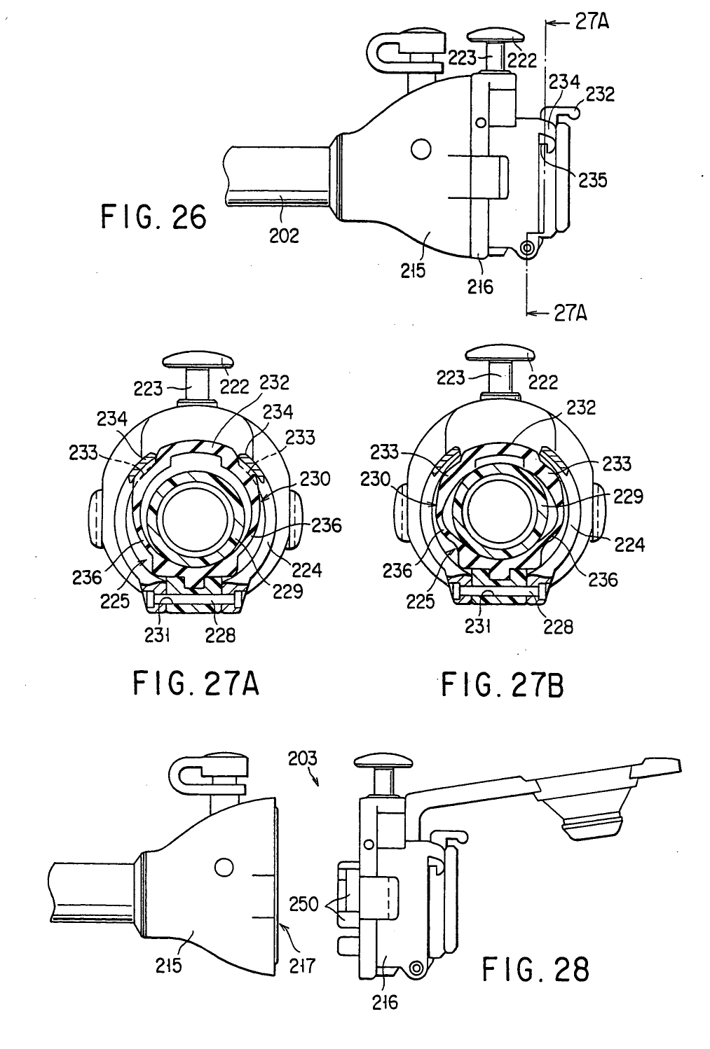

As shown in FIGS. 25 and 27A, the seal fixing portion 225 is composed of a fixing portion main body 229 and a deformed ring 230 fitted into the fixing portion main body 229. A hole 231 through which the rotary shaft 228 is passed is provided at the fixing portion main body 229. The deformed ring 230 has a deformed button portion 232, a pair of claws 233 provided on both sides of the deformed button portion 232, and a pair of thin wall portions 236 provided in the vicinity of the each claw 233. A pair of claws 233 are fitted into a groove 235 (refer to FIG. 26) inside of a pair of hangover portions 234 provided at the proximal end of the seal receiving portion 224 of the cover section 216 (refer to FIG. 26). FIG. 27B shows a state in which the deformed button portion 232 is held down. In this state, a pair of thin wall portions 236 of the deformed ring 230 is deformed, the upper part of the deformed ring 230 moves downward, and a pair of claws 233 are released from grooves 235 of a pair of hangover portions 234.

As shown in FIG. 25, the sealing member 226 consists of an elastic material. The sealing member is composed of a mounting portion 237 attached to the seal receiving portion 224; a main opening portion 238 present at its center of the mounting portion 237; an arm portion 239; and a subsidiary opening portion 240 present at the end of the arm portion 239. The arm portion 239 overhangs overhung on the side of the mounting portion 237 and then extends in the obliquely backward direction. A positioning shoulder portion 242 is provided over the full the periphery of the mounting portion 237. When the sealing member 226 is sandwiched between the seal receiving portion 224 and the seal fixing portion 225, the opening 208 of the holding portion 203; and an opening 241 of the seal fixing portion 225 and an opening 247 of the sealing member 226 are coaxial with each other by means of the positioning shoulder portion 242. Then, a cylinder portion 245 at the rear end of the seal fixing member 225 is fitted with a recess portion 244 formed between the shoulder portion 242 and a side wall 243 of the main opening portion 238. A sealing lip (a second sealing portion) 260 is provided at the end of the side wall 243 of the main opening portion 238. The sealing lip 260 abuts with the flap valve 219 in sealed state, and closes the opening 208 of the holding portion 203 in cooperation with the flap valve 219. In addition, a conical sealing film (a first sealing portion) 246 is provided at the main opening portion 238, and the opening 247 is present at its center of the sealing film 246. A thin film portion (a third sealing portion) 248 is provided at the subsidiary opening portion 240, and a small opening 249 with its internal diameter smaller than the opening 247 is provided at its center of the thin film portion 248. The sealing member 226 may be a duck bill valve or a slit valve.

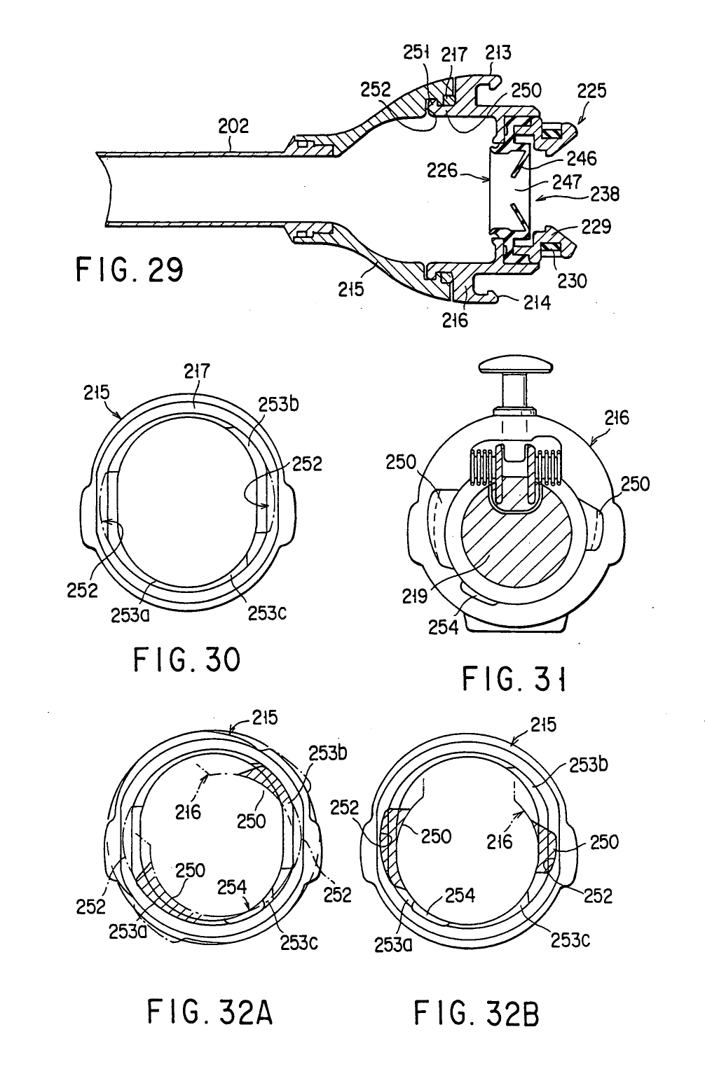

As shown in FIGS. 28 and 29, a pair of cam arms 250 extend from the tip end face of the cover section 216. A cam protrusion 251 protruding outwardly is provided at its tip end of each cam arm 250. A pair of cam grooves 252 which engage with the cam protrusion 251 are provided inside of the rear end face of the main body section 215.

A sealing ring 217 is provided on the rear end face of the main body section 215, and ensures air tightness when the main body section 215 and the cover section 216 are combined with each other.

FIG. 30 shows a rear end face of the main body section 215. As illustrated, two guide portions 253a and 253b are present inside of the sealing ring 217, and a pair of cam grooves 252 are positioned so as to be adjacent to the respective guide portions 253a and 253b and to be hidden at the back side of the sealing ring 217. A guide portion 253a is longer than the other guide portion 253b by the length of a long section 253c. As shown in FIG. 31, a protrusion 254 is provided on the tip end face of the cover section 216 so as to correspond to the long section 253c of the guide portion 253a.

Now, an operation of the above construction will be described.

After the needle portion 209 of the internal needle 204 has been inserted into the holding portion 203 and tube passage 206 of the sheath tube 1, thereby making the internal needle main body section 205 close to the holding portion 203 of the sheath tube 1, when the internal needle main body section 205 is pushed to the cover section 216 with aligning the orientation of the receiving portion 213 of the cover section 216 and the arm portion 211 of the internal needle main body section 205, the arm portion 211 is inclined inwardly, the claw 212 of the internal needle 204 is meshed over the claw 214 of the cover section 216. Then, the internal needle main body section 205 is connected and fixed to the cover section 216. Conversely, from this state, when the arm portion 211 of the main body section 205 is held down inwardly, the claws 212 and 214 are disengaged from each other, and the main body section 205 can be removed from the cover section 216.

From the state shown in FIG. 25, when the sealing member 226 (the main opening portion 238) is dropped in the seal receiving portion 224 of the cover section 216, and the seal fixing portion 225 is rotated to be pushed in the seal receiving portion 224, a pair of claws 233 of the deformed ring 230 abuts against a pair of hangover portions 234 provided at the seal receiving portion 224. At this time, a thin wall portion 236 of the deformed ring 224 is deformed by means of pushing force, the claw 233 moves downward to be over the hangover portion 234, and is fitted in the groove 235 inside of the hangover portion 234, and the sealing member 22.6 is fixed to the seal receiving portion 224. Conversely, when the deformed button 232 provided at the deformed ring 230 is pushed down while the sealing member 226 is fixed between the seal receiving portion 224 and the seal fixing portion 225, the claw 233 is removed from the groove 235. Then, the seal fixing portion 225 can be rotated, and the sealing member 226 can be removed from the seal receiving portion 224.

FIGS. 32A and 32B show a state in which a cam arm 250 and a protrusion 254 on the tip end face of the cover section 216 are combined with a cam groove 252 on the rear end face of the main body section 215, guide portions 253a and 253b. As shown in FIG. 32A, when the cover section 216 is fitted with the main body section 215 in the predetermined rotational position, a pair of cam arms 250 drop into a pair of guide portions 253a and 253b. At this time, the protrusion 254 drops into the long section 253c. In contrast in the case where the cover section 216 to be shifted by 180 degrees with respect to the main body section 215, the protrusion 254 is capable of dropping into the guide portion 253b, since the guide portion 253b does not have any section corresponding to the long section 253c. Thus, the cover section 216 cannot be fitted with the main body section 215. When the cover section 216 is rotated from the state shown in FIG. 32A, and a pair of cam arms 250 are brought to be coincident with a pair of cam grooves 252, the cover section 216 is completely connected to the main body section 215, as shown in FIG. 32B. As shown in FIG. 34, when the arm portion 239 of the sealing member 226 is bent, and the subsidiary opening portion 240 of the sealing member 226 is pushed to be fixed to the opening 241 of the seal fixing portion 225, the small opening 249 is arranged coaxially with the opening 247 and the tube passage 206 of the insert portion 203.

According to the sheath tube 201 of the present embodiment that has been described above, the following advantageous effects can be achieved.

(1) The sealing member 226 is removably mounted to the holding portion 203, and is rigidly fixed by the seal fixing portion 225. Specifically, the sealing member 226 is dropped into the seal receiving portion 224 of the cover portion 216, the seal fixing portion 215 is rotated to be dropped into the seal receiving portion 224, and the sealing member 226 can be fixed with one touch merely. Conversely the sealing member 226 can be removed merely by holding down the deformed button 232. Therefore, the sealing member 226 can be simply mounted to or removed from the holding portion 203, the sealing member 226 or the periphery of the seal receiving portion 224 of the cover section 216 on which this sealing member 226 is mounted is easily washed. In addition, in the case where the sealing member 226 is damaged and degraded, the sealing member 226 is easily replaced. In addition, even if a thick forceps or a stepped forceps is inserted or removed during operation, the sealing member 226 is free from slip-off.

(2) The sealing lip 260 coming into intimate contact with the flap valve 219 to ensure air tightness and the sealing film 246 coming into intimate contact with a medical instrument such as optical viewing tube or forceps to ensure air tightness are integrally formed. Therefore, the number of parts is reduced, and maintenance is facilitated. In addition, replacement cost can be reduced.

(3) The opening 241 provided at the seal fixing member 225 is arranged coaxially with the opening 247 of the main opening portion 238 of the sealing member 226, and the inclination of the medical instrument such as optical viewing tube or treatment instrument to be insert through the opening 247 is restricted. Therefore, a gap is not provided between the opening 247 and the medical instrument, and air tightness can be well ensured during surgery.

(4) The seal fixing portion 225 is turnably connected to the cover section 216 and cannot be separated from the cover section 216, and thus, there is no worrying about losing the seal fixing member 225.

(5) The internal needle 204 and the sheath tube 201 can be connected in one touch merely by pushing the main body section 205 of the internal needle 204 to the main body section 215 of the sheath tube 201. The internal needle 204 can be easily removed from the sheath tube 201 merely by pushing and bending the arm portion 211 inwardly.

(6) By means of the cooperative action of the protrusion 254 and the long section 253c of the guide portion 253a corresponding to the protrusion 254, the main body section 215 and the cover section 216 are free from being mounted in a reverse manner by 180 degrees.

(7) When the sealing member 226 is sandwiched between the seal receiving portion 224 and the seal fixing portion 225, the cylinder portion 245 at the rear end of the seal fixing portion 225 is fitted with a recess portion 244, and the shoulder portion 242 is engaged with the cylinder portion 245. Therefore, even if thick forceps is forced to be inserted or removed quickly, or even if thick forceps is drawn transversely, the sealing member 226 is free from moving or slip-off.

In this embodiment, the seal fixing portion 225 is rotatably attached to the cover section 216. However, the motion of the seal fixing portion 225 against the cover section 216 is limited to the rotation.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A trocar sheath tube comprising:
an insert portion introduced into the inside of a body, the insert portion having a tube passage, the tube passage being capable of passing a medical instrument internally, the insert portion guiding the medical instrument into the inside of a body through the tube passage;
a holding portion provided at the proximal end side of the insert portion in order to hold the proximal end side of the medical instrument inserted through the insert portion, the holding portion having an internal space communicating with the tube passage of the insert portion;
a sealing member removably attached to the holding portion, the sealing member having a first sealing portion coming into intimate contact with the medical instrument, the sealing member sealing a space between the medical instrument and the holding portion by the first sealing portion; and
a seal fixing frame removably mounted to the sealing member, the seal fixing frame adapted to fix the sealing member to the holding portion;
wherein an opening/closing valve for openably closing the internal space of the holding portion is provided in the holding portion;
wherein the sealing member has a second sealing portion which abuts with the opening/closing valve in a sealed state and closes the internal space of the holding portion in cooperation with the opening/closing valve; and
wherein the sealing member has a third sealing portion positioned to be spaced from the first sealing portion, the third sealing portion sealing a space between the medical instrument and the holding portion in intimate contact with the medical instrument.

2. A trocar sheath tube according to claim 1, wherein the seal fixing frame is turnably mounted to the holding portion.

3. A trocar sheath tube according to claim 1, wherein the opening/closing valve has a flap valve movable between a first position where the flap valve abuts with the second sealing portion and a second position where the flap valve is spaced from the second sealing portion.

4. A trocar sheath tube according to claim 3, wherein the flap valve is always biased toward the first position.

5. A trocar sheath tube according to claim 1, wherein the first and third sealing portions have holes through which the medical instrument can be inserted, respectively, and a diameter of the hole of the first sealing portion and that of the hole of the third sealing portion differ from each other.

6. A trocar sheath tube according to claim 1, wherein a shoulder portion engaged with a cylindrical portion of the seal fixing frame is provided at the sealing member.

7. A trocar sheath tube according to claim 1, wherein the sealing member is deformed by the seal fixing frame when the seal fixing frame contacts the sealing member.

8. A trocar sheath tube according to claim 1, wherein the sealing member has an arm portion for causing the first and third sealing portions to be coupled with each other.

9. A trocar sheath tube according to claim 8, wherein the arm portion is oriented in a longitudinal axial direction of the insert portion while the sealing member is mounted to the holding portion.

10. A trocar sheath tube according to claim 8, wherein the arm portion biases the third sealing portion toward a first position where the third sealing portion is arranged coaxially with the first sealing portion or toward a 5 second portion where the third sealing portion is distant from the first sealing portion by 180 degrees or over.

11. A trocar sheath tube according to claim 1, wherein the seal fixing frame has a hole for restricting the inclination of the medical instrument sealed by the first sealing portion of the sealing member.

12. A trocar sheath tube according to claim 1, wherein the sealing member has a portion sandwiched between the seal fixing frame and the holding portion.

13. A trocar sheath tube according to claim 1, wherein the first sealing portion of the sealing member has a hole through which the medical instrument can be inserted, and a diameter of the hole is changed by a hole diameter changeable member to be abutted with the sealing member.

14. A trocar sheath tube according to claim 5, wherein the diameter of the hole of the third sealing portion is smaller than that of the hole of the first sealing portion, and the holes of the first and third sealing portions are arranged in line along a longitudinal direction of the insertion portion.

15. A trocar sheath tube according to claim 14, wherein the third sealing portion is elastically deformable to enlarge the diameter of the hole of the third sealing portion.
